(19) United States
(12) Patent Application Publication
(10) Pub. No.: US 2008/0287673 A1
(43) Pub. Date: Nov. 20, 2008

(54) CEFDINIR PROCESS

(76) Inventor: Bandk PARTHASARADHI REDDY, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/570,256

(22) PCT Filed: Jun. 15, 2005
(86) PCT No.: PCT/IN05/00199

§ 371 Date: Dec. 8, 2006

(51) Int. Cl. C07D 501/60

(52) U.S. Cl. 540/220

(57) ABSTRACT

The present invention provides an improved process for the preparation of high assayed cefdinir. Thus, crude cefdinir is added to water at 25-30° C. and then 18% hydrochloric acid is slowly added to form a clear solution. The solution is cooled to −5° C. and stirred for 5 minutes at −5° C. to +5° C. Then temperature of the mass is raised to 35-38° C., stirred for 15 minutes at the same temperature. To the reaction mass eno carbon is added at 35-38° C. and stirred for 30 minutes at 35-38° C. Then the contents are filtered on hiflo bed and washed with water. The filtrate is then cooled to 25° C., the pH is slowly adjusted to 2.6 with saturated sodium bicarbonate solution and stirred for 60 minutes at 25-30° C. Filtered the solid, washed with water and dried at 40° C. under vacuum to give high assayed cefdinir.

### FIELD OF THE INVENTION

The present invention provides an improved process for the preparation of high assayed cefdinir.

### BACKGROUND OF THE INVENTION

U.S. Pat. No. 4,559,334, which is herein incorporated by reference, disclosed 7-substituted-3-vinyl-3-cephem compounds and their pharmaceutically acceptable salts; and their antimicrobial activity. Among them Cefdinir, chemically (6R,7R)-7-[[(2Z)-(2-amino-4-thiazolyl)(hydroxyimino)acetyl]amino]-3-ethenyl-8-oxo-5-thia-1-azabicyclo[4.2.0] oct-2-ene-2-carboxylic acid is a third generation cephalosporin antibiotic for oral administration and has a broader antibacterial spectrum than other orally administrable antibiotics. Cefdinir is particularly effective against staphylococci and streptococci. Cefdinir is represented by the following structure:

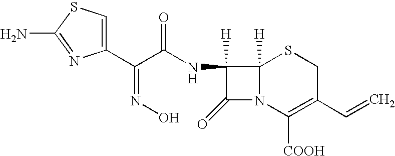

As per the process described for preparation of cefdinir in U.S. Pat. No. 4,559,334, cefdinir is obtained as amorphous solid. Various processes for preparing cefdinir are described in PCT Publication No. WO 2004/016623 A1, PCT Publication No. WO 2002/098884 A1 and U.S. Pat. No. 6,350,869 B1.

According to PCT Publication No. WO 2004/016623 A1, purified cefdinir is prepared by treating a solution of 7-[2-(2-aminothiazol-4-yl)-2-(methylcarbonyl oxyimino)acetamido]-3-vinyl-cephem-4-carboxylic acid in the form of salt, with p-toluenesulfonic acid, in methanol with concentrated sulfuric acid at or less than 10° C., adding aqueous sodium carbonate solution to adjust the pH to 5.0; subjecting the solution to carbon treatment and adjusting the pH of the filtrate to 3.0 with sulfuric acid solution to obtain cefdinir.

According to PCT Publication No. WO 02/098884 A1, cefdinir is prepared by treating p-TsOH.2DMAC salt of 7β-[(Z)-2-(2-aminothiazol-4-yl)-2-trityloxyiminoacetamido]-3-vinyl-3-cephem-4-carboxylic acid with formic acid and sulfuric acid to obtain pure cefdinir.

Cefdinir obtained by these processes has an important draw back of the being less assayed.

U.S. Pat. No. 6,350,869 B1 disclosed that cefdinir can form stable salt with dicyclohexylamine. Thus, according to the patent crude cefdinir can be purified by converting the crude cefdinir into cefdinir dicyclohexylamine salt, crystallizing pure cefdinir dicyclohexylamine salt, liberating the cefdinir from the salt by adding an acid and crystallizing pure cefdinir.

Even though the product obtained as above is high assayed cefdinir, the process is lengthy involving the additional steps of isolating salts such as dicyclohexylamine salt of cefdinir as crystalline solid as intermediates.

It has now been found that high assayed cefdinir can be produced in a simple manner and without the need for preparing crystalline cefdinir salts as intermediates.

According to the present invention, the high assayed cefdinir may be obtained by operationally convenient manner just by controlling temperature.

### DETAILED DESCRIPTION OF THE INVENTION

According to the present invention, there is provided a process for preparing high assayed cefdinir which comprises:

a) stirring an aqueous solution of a crude acid addition salt of cefdinir for at least about 3 minutes at −5 to +5° C.;
b) raising the temperature to about 33-40° C. and stirring for at least about 10 minutes at about 33-40° C.;
c) filtering the solution through hiflo bed at 25-40° C.;
d) adjusting the pH of the filtrate obtained to isoelectric point of cefdinir at 20-30° C. with a base to precipitate cefdinir from the solution; and
e) filtering or centrifuging the solid precipitated to obtain high assayed cefdinir.

Preferable acid addition salts are hydrochloric acid, phosphoric acid, sulfuric acid and methanesulfonic acid salt of cefdinir, more preferable acid addition salt being hydrochloric acid salt or methanesulfonic acid salt.

“Crude cefdinir” refers to cefdinir for which further treatment is required to obtain high assayed cefdinir.

“High assayed cefdinir” refers to cefdinir having the assay of not less than 95%, preferably between 96 and 101%.

The assay of cefdinir is determined by a suitable High Performance Liquid Chromatograph consisting of a pump an UV-VIS detector, sample injector, controller and integrator or equivalent software. The system is equipped with C₁₈ 5 μm 4.6×150 mm column (Inertial ODS 3 is suitable). Assay of cefdinir is performed by setting HPLC parameters like UV wavelength 247 nm, flow rate of about 1.0 ml/min., column temperature at about 30±2° C., and using Ammonium dihydrogen ortho phosphate buffer and water as a mobile phase in the ratio of 9:1.

The aqueous solution of crude acid addition salt of cefdinir used in step-(a) may be prepared, for example, by treating crude cefdinir with an acid in water medium or by dissolving crude acid addition salt of cefdinir in water.

The aqueous solution of crude acid addition salt of cefdinir may also be prepared by converting a sodium or potassium salt of cefdinir into corresponding crude acid addition salt of cefdinir by a conventional method in aqueous medium.

The aqueous solution is stirred preferably for 3 to 30 minutes, more preferably for 3 to 15 minutes at −5 to +50° C.

The temperature of the contents in step-(b) is raised to about 33-40° C., preferably to about 35-40° C. and stirred for at least about 10 minutes, preferably for 10 minutes to 1 hour, more preferably for 10 minutes to 30 minutes at about 33-40° C., and preferably at about 35-40° C.

The contents are then filtered in step-(c) through hiflo bed at 25-40° C., preferably at 33-40° C. and more preferably at 35-40° C. Optionally the contents obtained in step-(b) may be subjected to carbon treatment before step-(c) is carried out.

The pH of the filtrate obtained in step-(c) is adjusted to isoelectric point (step d) at about 20-30° C. with a base such as sodium bicarbonate or sodium carbonate to precipitate cefdinir from the solution.

The precipitated cefdinir is collected by filtration or centrifugation (step e) to obtain high assayed cefdinir.

The following examples are given for the purpose of illustrating the present invention and should not be considered as limitations on the scope or spirit of the invention.

### EXAMPLE 1

### Step-I

2-Amino-alpha-(acetyloxyimino)-4-thiazole acetic acid (76 gm) is added to methylene chloride (1150 ml) at 25-35° C., distilled to collect 30 ml of methylene chloride at 40° C., refluxed for 30 minutes at 40° C. and again distilled to collect 110 ml of methylene chloride. Then the reaction mass is cooled to 15° C., bis[thio(benzothiazole)] (167 gm) and triphenyl phosphine (137.3 gm) are added for 5 minutes at 15° C. and stirred for 10 minutes at the same temperature. To the reaction mass, triethyl amine (50.8 gm) is added for 5 minutes at 15-20° C. and stirred for 30-120 minutes at the same temperature. Filtered the solid immediately and washed with methylene chloride (300 ml) to give 110 gm of wet compound.

To the wet compound, methylene chloride (700 ml) is added at 25° C. and stirred for 1 hour at 15-20° C. Filtered the solid, washed with methylene chloride (300 ml) and dried under vacuum at 50° C. to give 97 gm of 2-mercapto-1,3-benzothiazolyl-(Z)-2-(2-aminothiazol-4-y)-2-(acetyloxyimino)acetate (HPLC Purity: 92%, Moisture content: 0.6% w/w).

### Step-II

To the mixture of 7-amino-3-vinyl-3-cephem-4-carboxylic acid (40 gm) and tetrahydrofuran (400 ml), 2-mercapto-1,3-benzothiazolyl-(Z)-2-(2-aminothiazol-4-yl)-2-(acetyloxyimino)acetate (79 gm) and water (200 ml) are added at 25° C. and then the contents are cooled to 15-20° C. To the contents, triethyl amine (20 gm) is slowly added for 40 minutes at 15-20° C. (pH: 8.0-8.5) and stirred for 3-4 hours at the same temperature. Methylene chloride (400 ml) is added to the reaction mass at 15-20° C., stirred for 15 minutes, water (200 ml) is added and stirred for 15 minutes. Then separated the layers and the aqueous layer is washed two times with methylene chloride (each time 200 ml). Combined the organic layer and extracted with water (200 ml). Combined the aqueous layer, carbon (4 gm) is added at 20° C. and degassed for 30 minutes. The resulting mass is filtered on hiflo bed, washed with 200 ml of water and then the pH of the aqueous layer is adjusted to 8.1 to 8.5 with potassium carbonate solution (32 gm in 164 ml water). To the aqueous layer, ammonium chloride (26.4 gm) is added at 20-25° C. while maintaining the pH 8.0-8.2 with 20% potassium carbonate and stirred for 25 minutes at the same temperature. Then the temperature of the mass is raised to 35-40° C., pH of the solution is adjusted to 2.4-2.5 with 9N sulfuric acid at 35-40° C. and stirred for 2 hours at the same temperature. Filtered the solid, washed with water (400 ml) and dried at 40-45° C. under vacuum to give 59 gm of crude cefdinir (HPLC Purity: 99.2, Assay: 86%).

### Step III

Crude cefdinir (20 gm) is added to water (300 ml) at 25-30° C. and then 18% hydrochloric acid (60 ml) is slowly added to form a clear solution. The solution is cooled to −5° C. and stirred for 5 minutes at −5° C. to +5° C. Then ternperature of the mass is raised to 35-38° C., stirred for 15 minutes at the same temperature. To the reaction mass eno carbon (4.0 gm) is added at 35-38° C. and stirred for 30 minutes at 35-38° C. Then the contents are filtered on hiflo bed and washed with water (40 ml). The filtrate is then cooled to 25° C., the pH is slowly adjusted to 2.6 with saturated sodium bicarbonate solution and stirred for 60 minutes at 25-30° C. Filtered the solid, washed with water (80 ml) and dried at 40° C. under vacuum to give 11.2 gm of cefdinir (HPLC Purity: 99.7%, Assay: 96.8%).

### EXAMPLE 2

Crude cefdinir (20 gm, obtained in step-II of example 1) is added to water (300 ml) at 25° C. and then 1:1 methanesulfonic acid (68 ml) is added at 25-28° C. to form a clear solution. The solution is cooled to 0-5° C., stirred for 10 minutes at 0-5° C. and again the temperature is raised to 35° C. Eno carbon (4 gm) is added to the reaction mass at 30-35° C., stirred for 30 minutes and filtered through hiflo bed. Hiflo bed is washed with water (40 ml), the pH of the filtrate is adjusted to 2.6 with saturated sodium bicarbonate solution at 25° C. and stirred for 1 hour at 25° C. Filtered the solid, washed with water (50 ml) and dried at 45° C. under vacuum to give 10.4 gm of cefdinir (HPLC purity: 99.4%, Assay: 96.5%).

1. A process for the preparation of high assayed cefdinir which comprises:
a) stirring an aqueous solution of an acid addition salt of crude cefdinir for at least about 3 minutes at −5 to +5° C.;
b) raising the temperature to about 33-40° C. and stirring for at least about 10 minutes at about 33-40° C.;
c) filtering the solution through a hiflo bed at 25-40° C.;
d) adjusting the pH of the filtrate obtained to the isoelectric point of cefdinir at 20-30° C. with a base to precipitate cefdinir from the solution; and
e) filtering or centrifuging the solid precipitated to obtain high assayed cefdinir.

2. The process as claimed in claim 1, wherein the high assayed cefdinir obtained in step-(e) has an the assay of not less than 95%.

3. The process as claimed in claim 2, wherein the assay of cefdinir is between 96% and 101%.

4. The process as claimed in claim 1, wherein the acid addition salt is selected from the salts of cefdinir with hydrochloric acid, phosphoric acid, sulfuric acid and methanesulfonic acid.

5. The process as claimed in claim 4, wherein the acid addition salt is the salt of cefdinir with hydrochloric acid or methanesulfonic acid.

6. The process as claimed in claim 1, wherein the aqueous solution in step-(a) is stirred for 3 to 30 minutes at −5 to +5° C.

7. The process as claimed in claim 6, wherein the aqueous solution is stirred for 3 to 15 minutes at −5 to +5° C.

8. The process as claimed in claim 1, wherein the temperature of the contents in step-(b) is raised to about 35-40° C.

9. The process as claimed in claim 1, wherein the contents in step-(b) are stirred for 10 minutes to 1 hour at about 35-40° C.

10. The process as claimed in claim 9, wherein the contents are stirred for 10 minutes to 30 minutes at about 35-40° C.

11. The process as claimed in claim 1, wherein the contents in step-(c) are filtered through a hiflo bed at about 33-40° C.

12. The process as claimed in claim 11, wherein the contents in step-(c) are filtered through a hiflo bed at about 35-40° C.

13. The process as claimed in claim 1, wherein the contents obtained in step (b) are subjected to carbon treatment before step-(c) is carried out.

14. The process as claimed in claim 1, wherein the base in step-(d) is sodium bicarbonate or sodium carbonate.
